(19) United States
(12) Patent Application Publication
(10) Pub. No.: US 2011/0015490 A1
(43) Pub. Date: Jan. 20, 2011

(54) METHOD AND SYSTEM FOR CANNULA POSITIONING

(75) Inventors: Karen I. Trovato, Putnam Valley, NY (US); Nicholas D. Marchuk, Evanston, IL (US); Aleksandra Popovic, New York, NY (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/921,575

(22) PCT Filed: Mar. 3, 2009
(86) PCT No.: PCT/IB09/50884

§ 371 Date: Sep. 9, 2010

Related U.S. Application Data
(60) Provisional application No. 61/038,225, filed on Mar. 20, 2008.

(51) Int. Cl. A61B 1/32

(52) U.S. Cl. 600/227

(57) ABSTRACT

An active cannula (10) can include a plurality of hollow tubes (100, 110, 120), a plurality of blocks (200, 210, 220), and a track (1800). Each of the blocks can be connected to one of the hollow tubes. Each of the blocks can be operably connected to the track for movement therealong. In a first position, the blocks can be separate from each other along the track and the plurality of hollow tubes can be nested. In a second position, the blocks can be adjacent to each other along the track and the plurality of hollow tubes can be extended. In the second position, the plurality of hollow tubes can provide access to the targeted anatomical region from outside of the body. Other embodiments are disclosed.

### FIELD OF THE INVENTION

This disclosure relates generally to medical systems and more specifically to a method and system for cannula positioning.

### BACKGROUND

The use of minimally invasive surgical procedures has grown in recent years due to their ability to allow for monitoring or surgical treatment without the trauma typically resulting from open surgery. Minimally invasive surgical procedures can also allow for access to anatomical regions that were previously unreachable.

Typical tools utilized in minimally invasive surgical procedures can include rigid laparoscopic devices, robotic devices, or scopes that utilize marionette-like strings for control. Each of these devices imposes certain limitations and has inherent drawbacks. For instance, rigid laparoscopic devices can require open space for maneuvering both inside and outside the body. This space requirement can preclude the use of rigid laparoscopic devices in many types of procedures.

Robotic devices are unable to reach far into the human body since they rely on motors to control each joint angle. Motors are often large compared to the small anatomical spaces of the body. The number of robotic joints limits the complexity of the environment through which the robot can reach. Robots are often six degrees of freedom so that they can reach a fixed point in freespace at a particular orientation. The addition of anatomical obstacles effectively reduces the remaining active degrees of freedom. Additional motors to increase dexterity, also add weight and size. For example, robotic devices having seven degrees of freedom are often heavy and frequently hard to control smoothly.

Scopes that are controlled by marionette-like strings, such as bronchoscopes and endoscopes rely on the marionette strings to control the distal part of the scope. Although thinner than a robotic device, control of only one arc at the distal end of the scope is also a significant limitation. Further, the use of marionette-like strings requires an additional increase in device radius.

Active cannulas have been developed where the cannula is made from several concentric, pre-curved, superelastic tubes. Each tube can telescope in and out of the others, and can also be spun. Interaction and manipulation of the tubes can be utilized by the physician for positioning the distal end of the tubes in the desired position. However, achieving the correct orientation of the tubes can be difficult, inaccurate and time consuming. Manual assembly can also be difficult and time consuming. The tubes can be hard to handle, particularly at their smaller sizes.

Accordingly, there is a need for a cannula, such as an active cannula, that can access difficult to reach anatomical regions. There is a further need for a cannula, such as an active cannula, that is easy to control. There is yet a further need for a method and system for manufacturing a cannula, such as an active cannula.

### SUMMARY

The Summary is provided to comply with 37 C.F.R. §1.73, requiring a summary of the invention briefly indicating the nature and substance of the invention. It is submitted with the understanding that it will not be used to interpret or limit the scope or meaning of the claims.

Exemplary embodiments according to inventive aspects of the present disclosure can include a guide or other device that allows for movement of the cannula tubes from a nested position to an extended position. The exemplary embodiments can include structure or techniques that configure the cannula tubes as to length and or orientation so that a desired path can be followed to reach a targeted anatomical region.

In one exemplary embodiment of the present disclosure, a device for accessing a targeted anatomical region of a body is provided. The device can include a plurality of hollow tubes, a plurality of blocks, and a track. Each of the blocks can be connected to one of the hollow tubes. Each of the blocks can be operably connected to the track for movement therealong. In a first position, the blocks can be separate from each other along the track and the plurality of hollow tubes can be nested. In a second position, the blocks can be adjacent to each other along the track and the plurality of hollow tubes can be extended. In the second position, the plurality of hollow tubes can provide access to the targeted anatomical region from outside of the body.

In another exemplary embodiment, a system for accessing a targeted anatomical region of a body is provided. The system can include a plurality of support structures; a plurality of tubes that are each connected to one of the support structures; and a guide. Each of the support structures can be operably connectable with the guide. The guide can allow movement of at least a portion of the plurality of support structures therealong. The plurality of tubes can be nested when the plurality of support structures are in a first position along the guide. The plurality of tubes can be extended when the plurality of support structures are in a second position along the guide. In the second position, at least one of the plurality of tubes can access the targeted anatomical region of the body.

In a further exemplary embodiment, a method for accessing a targeted anatomical region of a body is provided. The method can include determining a path to the targeted anatomical region; providing a plurality of tubes having a length and shape to follow the path; connecting each of the tubes to support structures; positioning the support structures so the tubes are in a nested position; and moving the support structures so the tubes are in an extended position and a portion of the plurality of tubes is in proximity to the targeted anatomical region.

The technical application includes, but is not limited to, facilitating surgical procedures by providing easy access to difficult to reach anatomical regions. The technical effect further includes, but is not limited to, facilitating the manufacture of, and/or control over, cannulas, such as active cannulas.

The above-described and other features and advantages of the present disclosure will be appreciated and understood by those skilled in the art from the following detailed description, drawings, and appended claims.

### BRIEF DESCRIPTION OF THE DRAWINGS

FIG. 1 is a schematic illustration of a pair of tubes of an active cannula according to an exemplary embodiment of the present invention;

FIG. 2 is a schematic illustration of supporting blocks with the tubes of FIG. 1 according to an exemplary embodiment of the present invention;

FIG. 3 is a schematic illustration of a configuration device for connecting the tubes and supporting blocks of FIG. 2 according to an exemplary embodiment of the present invention;

FIG. 4 is another schematic illustration of the configuration device of FIG. 3;

FIG. 5 is a schematic illustration of one of the supporting blocks of FIG. 2;

FIG. 6 is another schematic illustration of the supporting block of FIG. 5;

FIG. 7 is an exploded schematic illustration of one of the supporting blocks and tubes of FIG. 2;

FIG. 8 is a schematic illustration of the supporting block and the tube of FIG. 7;

FIG. 9 is a cross-sectional schematic illustration of the supporting block and the tube of FIG. 7;

FIG. 10 is a schematic illustration of a portion of the configuration device of FIG. 3;

FIG. 11 is a schematic illustration of another portion of the configuration device of FIG. 3;

FIG. 12 is a schematic illustration of another portion of the configuration device of FIG. 3;

FIG. 13 is a schematic illustration of another portion of the configuration device of FIG. 3;

FIG. 14 is a schematic illustration of another portion of the configuration device of FIG. 3;

FIG. 15 is a schematic illustration of another portion of the configuration device of FIG. 3;

FIG. 16 is an exploded schematic illustration of the tubes of FIG. 1 with block adapters according to an exemplary embodiment of the present invention;

FIG. 17 is an exploded schematic illustration of a portion of the tubes and adapters of FIG. 16;

FIG. 18 is a schematic illustration of an active cannula in a nested position according to an exemplary embodiment of the present invention;

FIG. 19 is a schematic illustration of the active cannula of FIG. 18 in an extended position;

FIG. 20 is a schematic illustration of the active cannula of FIG. 18 in use with a patient; and

FIG. 21 is a schematic illustration of an active cannula according to another exemplary embodiment of the present invention.

### DETAILED DESCRIPTION OF THE INVENTION

The exemplary embodiments of the present disclosure are described with respect to minimally invasive surgery of a human. It should be understood by one of ordinary skill in the art that the exemplary embodiments of the present disclosure can be applied, whether human or animal. In one exemplary embodiment of the present invention, the active cannula can build the intended motion into the construction of the device so that motors, wires or other control structure is unnecessary, and these small, thin devices are able to reach far into the human anatomy. The active cannula of the present invention can be configured to reach a target, while avoiding anatomical obstacles.

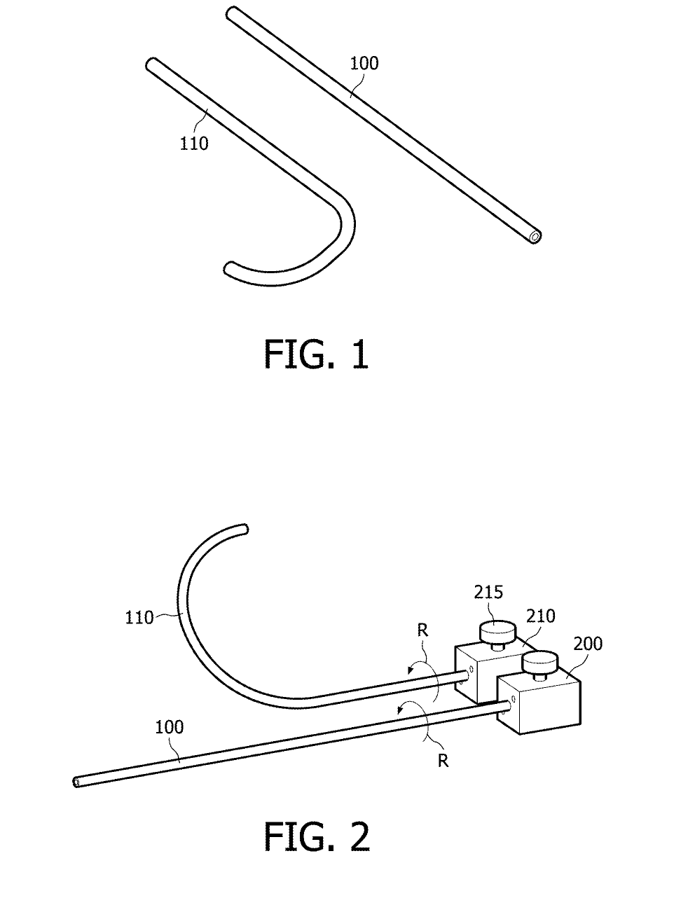

Referring to the drawings, and in particular to FIG. 1, a series of tubes 100, 110 are shown. Tubes 100, 110 can have particular dimensions and shapes so that when nested together and then extended from their nested position, they travel to a targeted anatomical region. In one embodiment, the inner and outer diameters of the tubes can be chosen to facilitate deployment from the nested position to the extended position, while maintaining a desired overall shape so that the tubes can travel to the targeted anatomical region. While the exemplary embodiments are described with respect to the use of cylindrical tubes 100, 110, the present disclosure contemplates the use of other shapes of tubes, including an oval cross-section or other shape that can facilitate the passage of a particular tool or other device therethrough.

The exemplary embodiments describe a starting position of the tubes for a cannula procedure as being a nested position. It should be understood by one of ordinary skill in the art that the nested position can include a completely nested position where each of the inner tubes are at least substantially within an outer tube and can be a partially nested position where some portion of one or more of the inner tubes are within an outer tube (including partially extending outside of the outer tubes). The nested position can facilitate extension of the tubes by easing their movement with respect to each other rather than requiring them to be separately threaded with each other during the procedure.

The tubes 100, 110 can be made from various materials or combinations of materials. In one embodiment, the tubes 100, 110 can be made from a shape memory alloy (SMA), such as nickel titanium (e.g., Nitinol), which can be deformed while remembering its shape. The tubes 100, 110 can be any number of telescoping, pre-shaped flexible SMA tubes that are extended along the anatomy with a particular shape or curvature. The use of SMA tubes and their ‘memory’ enables each of the inner telescoped tubes to straighten or conform into the larger tube surrounding it until extended.

In one embodiment, one or more of the tubes 100, 110 can be made, in whole or in part, from Shape Memory Polymers (SMPs). SMPs have the ability of shape preservation, similar to Nitinol. SMPs have a transition temperature, i.e., the temperature at which the shape preservation takes place, that can provide for use in various environments. For instance, the tubes 100, 110 made from SMP can have a wide range of transition temperatures (e.g., between −75° C. and +75° C.). SMPs can have other advantages including: cost effective, such as about 10% lower than the price of Nitinol; highly reusable, such as allowing 500 shape memory/recovery cycles; shape recovery of 400% as compared to 7-8% in conventional metal shape alloys; able to be sterilized, biocompatible and biodegradable.

In one embodiment, SMP microtubes (e.g., 250 μm and larger) can be utilized. For instance, SMP microtubes are described in U.S. Pat. No. 6,059,815 to Lee, the disclosure of which is hereby incorporated by reference. Commercially available SMP tubes that can be used herein are available from Memry Inc. of Bethel, Conn. and MnemoScience GmbH of Aachen, Germany.

While the exemplary embodiments are described with respect to the use of nested tubes that can be extended to allow for access to a targeted anatomical region by passing a tool or other device through the extended tubes, the present disclosure contemplates the inner most tube including a tool or other device. For example, the inner most tube can have an imaging device at an end thereof so that when extended from the nested position the imaging device is positioned in the targeted anatomical region. In one embodiment, the inner most tube can have a closed end, such as a fiber optic line for transmitting light to and/or from the targeted anatomical region. In another embodiment, the inner most tube can be partially or completely solid.

In one embodiment, a target can be selected within a specific anatomical area and a series of SMA tubes can be created with specific relative orientation and length for each in order to travel to and reach the target from a nested position to an extended position. For example, the following criteria can be selected for creating SMA tubes to reach a particular anatomical region:

| Tube Type | Orientation(if any) | Length |
|---|---|---|
| Straight | | 60 mm |
| 28 mm Curved | CCW 45 degrees | 12 mm |
| Straight | | 17 mm |
| 28 mm Curved | CW +90 degrees | 7 mm |

Each tube in the series can be a straight, curved or other shaped component tube of known length, such as the exemplary component tubes 100, 110. Component tubes can be made in any shape, including being straight or having a partial or full arc. Various degrees of curvature can be utilized. For example, curvature of 180 degrees can be utilized for one or more of the SMA tubes, such as where the targeted anatomical region is in the lungs since airway structures rarely curve beyond this degree in one continuous arc. Additional shapes, including a helix, can also be utilized for the SMA tubes.

Referring additionally to FIG. 2, each of the tubes 100, 110 can have a block or support structure 200, 210 connected thereto. The blocks 200, 210 can be securely connected at a specific distance along the tubes 100, 110, and securely connected at a particular orientation of the tubes (as shown by Arrows R in FIG. 2). The tubes 100, 110 can be nested and the blocks 200, 210 can be set into a track or other guide structure, as described more particularly below, which allows them to be deployed, including manual deployment, according to the cannula configuration specified by the output of a planner. While the exemplary embodiments are described with respect to the use of rectangular blocks 200, 210, the present disclosure contemplates the use of other shapes of support structures that are connected to each of the tubes 100, 110. The support structures (e.g., blocks 200, 210) allow for fixing of a desired length and orientation of each of the tubes so that when deployed from a nested to an extended position, the tubes can follow a desired path to the targeted anatomical region.

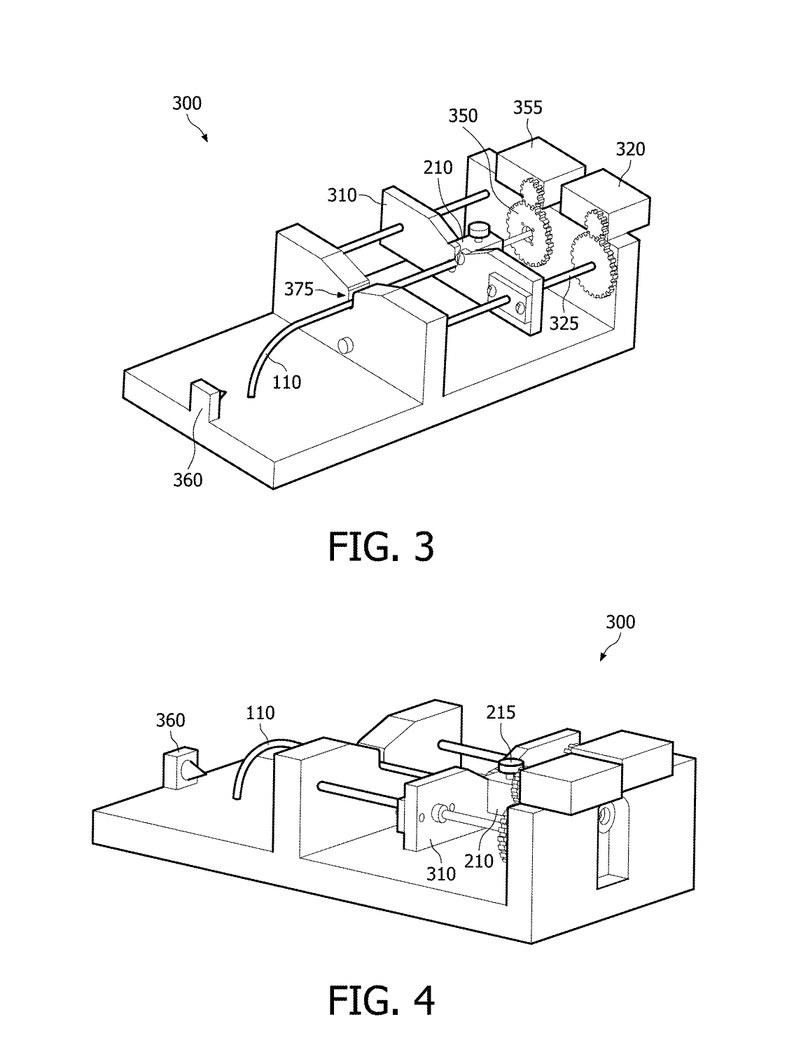

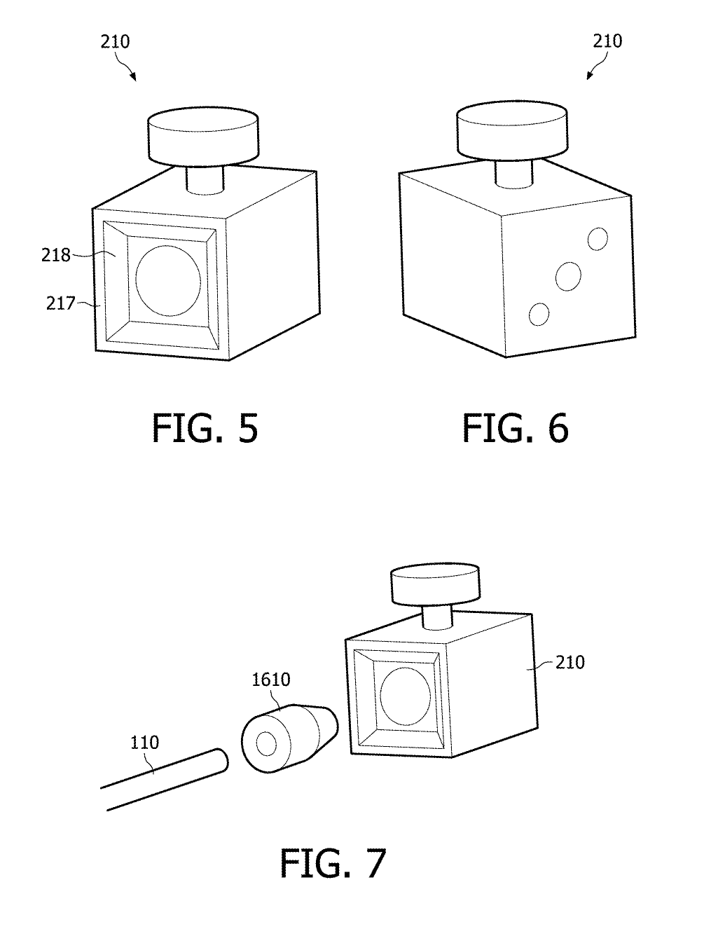

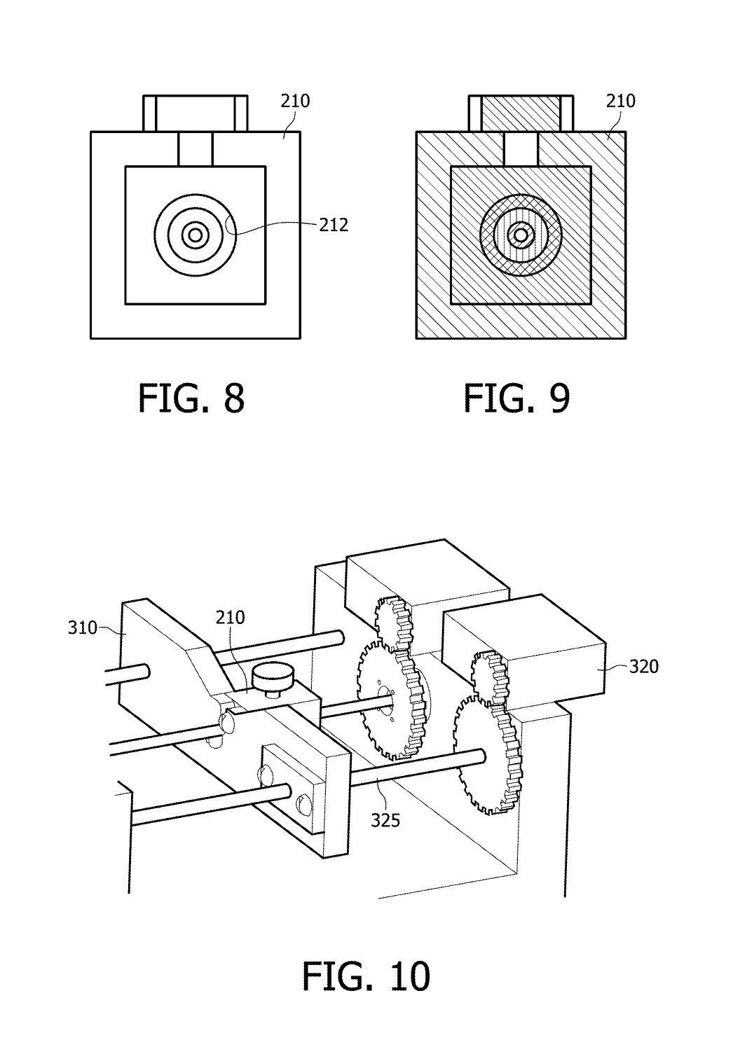

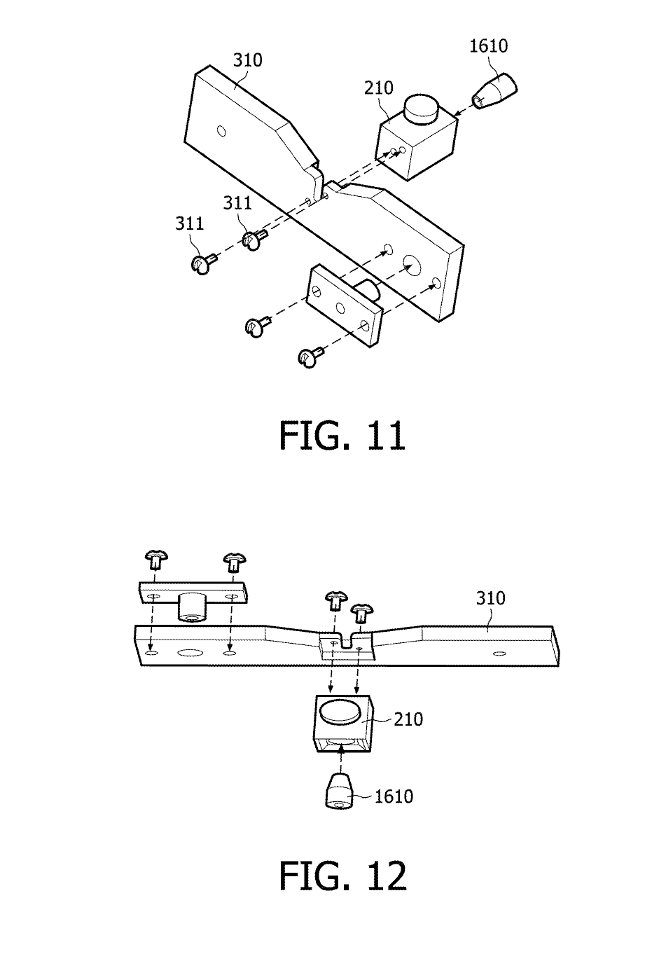

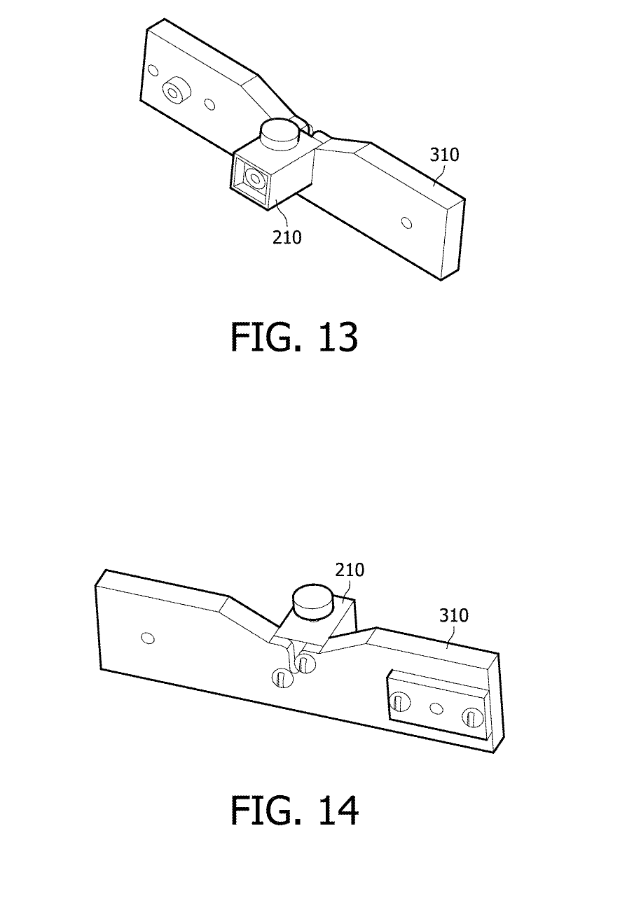

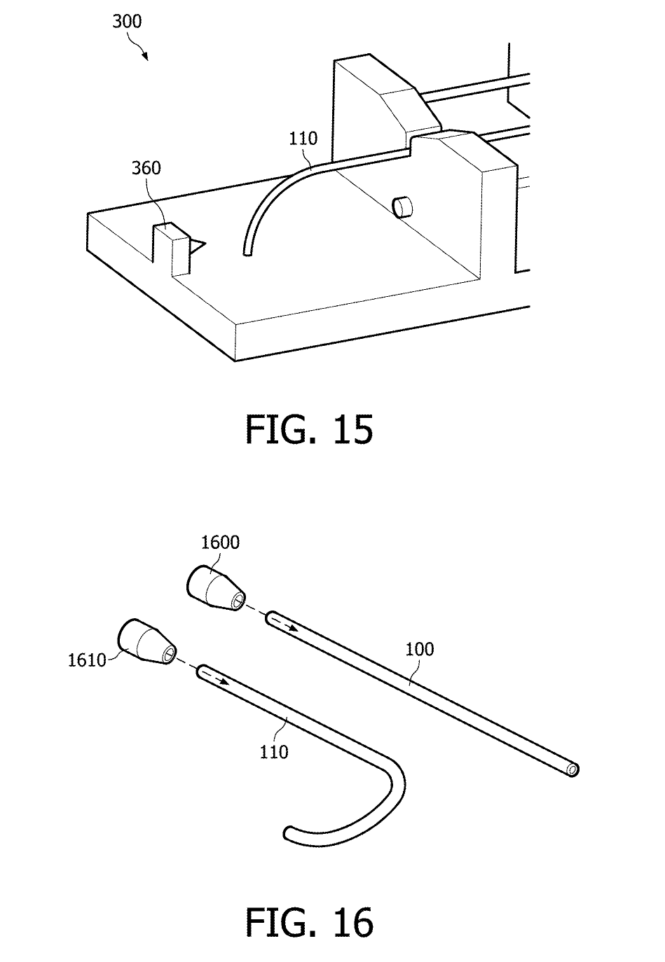

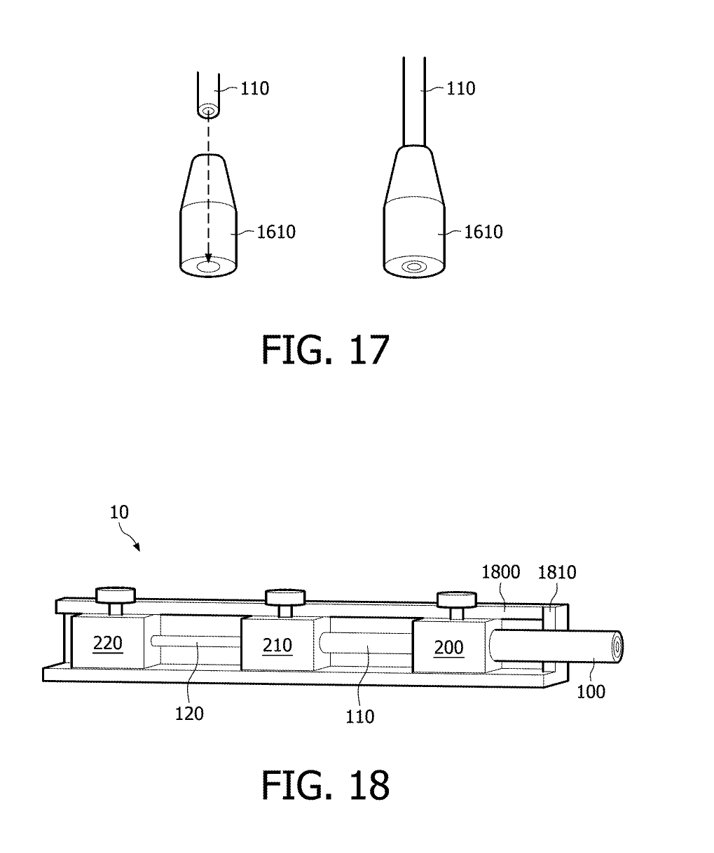

Referring additionally to FIGS. 3 through 17, a configuration device 300 can be used to set up a block or other support structure, such as the block 210, that is attached to a SMA tube, such as the tube 110. The device 300 can include a moveable plate 310 or other structure, which is a translation mechanism to set the length of the tube 110 with respect to the block 210. In one embodiment, a servo motor 320 can turn a lead screw 325 to a desired rotation resulting in the plate 310 moving to a desired distance along the device 300 while the tube remains stationary. The present disclosure also contemplates other components, devices and configurations for moving the plate 310 along the device 300, including manually moving the lead screw, such as with a turn knob. Movement of the plate 310 results in movement of the tube 110 with respect to the block 210 so that a desired length of tube can be obtained. The present disclosure also contemplates moving the tube 110 while the block 210 or other support structure remains stationary.

To obtain a desired orientation of the tube 110 with respect to the block 210, a rotation device 350 and a calibration mechanism 360 can be included in the device 300. The rotation device 350 can be adjusted by a servo motor 355 or other adjustment mechanism (including manual adjustment). The rotation device 350 can rotate the tube 110 with respect to the block 210 to a desired orientation, such as based on a calibration achieved by the calibration mechanism 360. For example, the calibration mechanism 360 can have a laser to define the zero orientation. The light from the laser can highlight the tube 110 when it is in the nominal (e.g., zero) orientation.

In one embodiment of a method of manufacture, an adapter 1600, 1610 can be attached to the end of the tube 100, 110, such as through use of adhesive (e.g., LOCTITE® glue). If the tube 110 has a curved end, the adapter 1610 can be placed on the straight end as shown in FIG. 16. In one embodiment, the end of the tube 100, 110 can be made flush with the end of the adapter 1600, 1610, such as through alignment on a flat surface.

To achieve the desired length of tube 110, the tip of the tube can be threaded through the opening of the adapter 1610, such as while it is inside the block 210. The tube 110 can be rested across the support slot 375 of the device 300 on one end and be fitted with the rotation device 350 on the other end. The adapter 1610 can be secured in the block 210 such as through a set screw 215. Other locking structures or techniques can also be used, such as a ratchet or lug structure. The servo motor 320 can then turn the drive screw 325 until the plate 310 and the block 1610 is moved with respect to the tube 110 to the desired length, such as specified by the planner. Screws 311 or other connection devices can be used to temporarily connect the block 210 with the plate 310 for movement thereof.

For tubes having a curvature where the orientation must be set, then the tube can be set into the block at a specific orientation. The tube 110 can be rotated until the tip is at a known orientation, such as through use of the calibration mechanism 360. While the exemplary embodiment describes the laser of the calibration mechanism 360 being below the support slot 375, the present disclosure contemplates the laser being on a parallel fixed structure. When the tube 110 rotates within the laser line, the tube can reflect the light from the laser. The orientation servo 355 can then be calibrated to that angle. The orientation servo 355 can then rotate the tube 110 to the desired orientation or angle specified by the planner.

Once the desired length and the desired orientation have been obtained, the tube 110 can be secured to the adapter 1610, such as through adhesive placed along the edge of the adapter. In one embodiment, the block 210 can have an opening with an inward taper 212 so that the adhesive remains below the surface of the block. Any excess tubing extending beyond the back 217 of the block 210 can be cut away. This ensures that each block can be moved snugly against the next block. In one embodiment, a bevel or cone-shaped space 218 can be used to enable the tube 110 to be cut below the outer most surface of the back 217 to minimize any gap between adjacent blocks.

Referring additionally to FIGS. 18-20, once all of the required tubes 100, 110, 120 are set into their respective blocks 200, 210, 220, the tubes can be threaded or nested into one another in sequence. For example, if the tubes 100, 110, 120 extend to the right as in FIG. 18, the blocks 200, 210, 220 can be arranged from the largest diameter to the smallest diameter tube. The blocks 200, 210, 220 can then be positioned within a track 1800 or other guide.

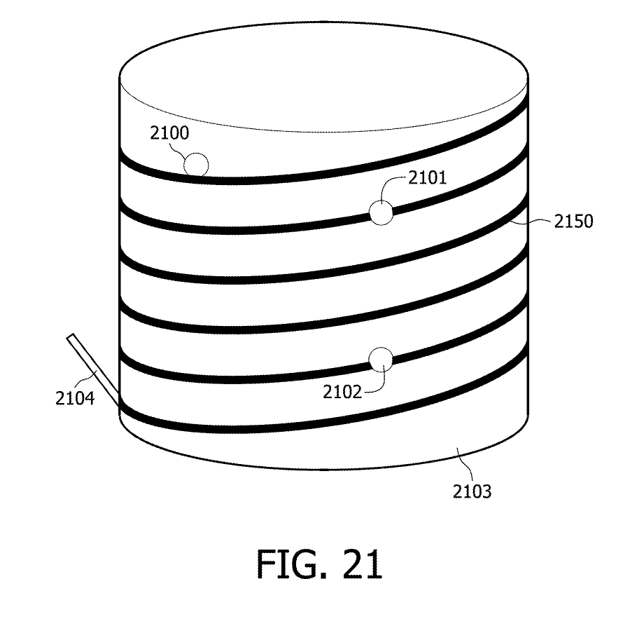

The exemplary embodiment of active cannula 10 shows the track 1800 as a straight sliding device containing slideable blocks. However, it should be understood by one of ordinary skill in the art that alternative shapes of the track 1800 and/or paths for the slideable blocks can be utilized. For example and referring to FIG. 21, a coil shaped path can be utilized, such as to save space and enable easier handling. In one embodiment, knobs 2100, 2101, and 2102 can be slideably moved along slots 2150 and lockable in a variety of locations. The slots 2150 can be formed in a cylindrical structure 2103, can have marks along them to provide calibrated distances for each of the knobs, and can provide for movement of the tubes 2104 along the desired path.

Referring back to FIGS. 1-20, in operation, the active cannula 10 can be positioned in proximity to the patient, such as along the side of the patient as shown in FIG. 20. In one embodiment, the track 1800 can be connected to a bed or other patient support and the blocks 200, 210, 220 and tubes 100, 110, 120 can then be positioned along the track 1800. When the tubes 100, 110, 120 are deployed, the largest tube 100 can be placed into the patient. In one embodiment, the largest tube 100 can be a flexible tube that retains its deformed shape so that it can be adjusted into position in or near the patient. In the exemplary embodiment, the active cannula 10 can be positioned through the mouth of the patient, but one of ordinary skill in the art would recognize that other points of entry can also be used for reaching the targeted anatomical region, such as the nostrils.

To reach the targeted anatomical region, the block 200 connected to the largest or outer most tube 100 can be advanced along the track 1800, such as until it is against a jam 1810 of the track. The next block 210 can then be advanced along the track 1800 until it abuts against the block 200 so that the tube 110 advances and extends from the tube 100. The third block 220 can then be advanced along the track 1800 until it abuts against the block 210 so that the tube 120 advances and extends from the tube 110. Other blocks and tubes (not shown) can be similarly moved along the track 1800. The track 1800 can restrict movement of the blocks in all but two directions that are opposite to each other so that a path can be followed by the tubes as they are extended from their nested position. Once all the tubes 100, 110, 120 are fully extended, the targeted anatomical region should be reached by a distal end of the smallest of the tubes (e.g., tube 120). The exemplary embodiment of the active cannula 10 shows three blocks 200, 210, 220 and three tubes 100, 110, 120 that are used to reach the targeted anatomical region, but the present disclosure contemplates any number of blocks and tubes being used to travel along a desired path and extend into the targeted anatomical region.

The exemplary embodiment shows the tubes fully extended to reach the target location. However, the present disclosure contemplates for partial extension, including tubes with marks along their length or the track 1800 may have marks along its length so that intermediate locations can be achieved. For instance, a planner can provide the marker values that lock each block so that a second (or more) location can be reached with the same set of tubes. Re-use of the same tubes for one or more alternate locations can save time and is cost efficient, as opposed to requiring multiple cannulas to reach multiple positions.

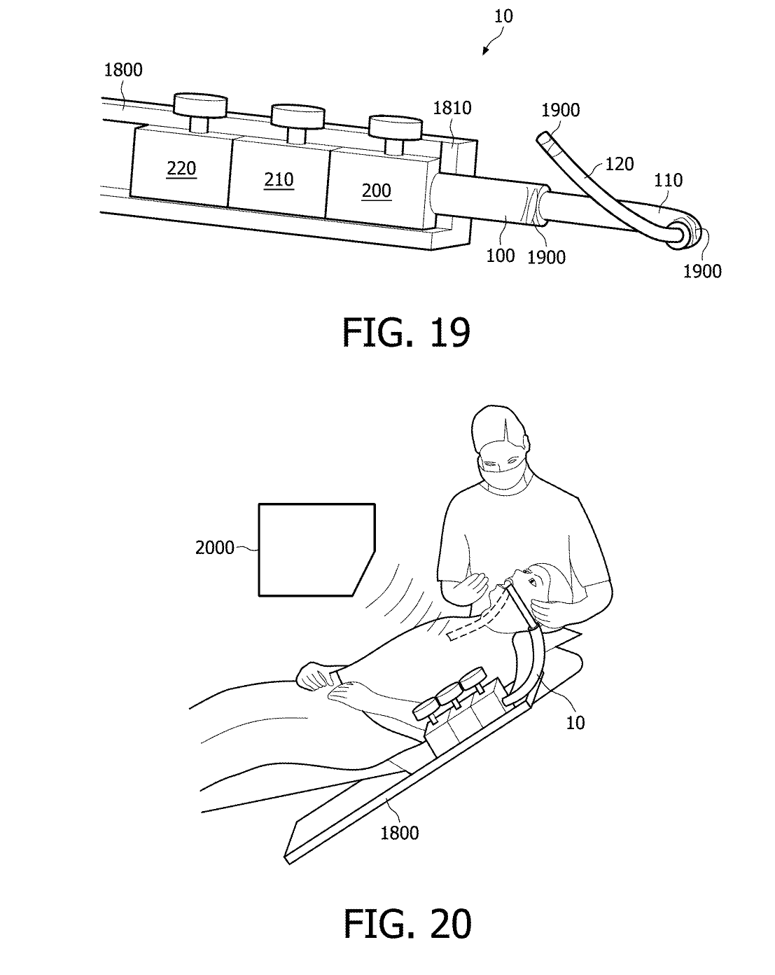

A fully extended active cannula is shown in FIG. 19. As each of the tubes extends from its nested position due to movement of its block, the tube travels along a desired path, which can be linear or non-linear, due to the length and shape of the tube. In one embodiment, the use of SMA tubes allows the tubes to each transition back to their desired shape from their deformed shape as they are extended from their nested position. The nesting of each of the tubes provides a temporary deformation to non-linear SMA tubes. The particular path that is to be followed can be determined or otherwise obtained based on a number of techniques, including measurements of the patient, imaging, known paths, and the like.

In one embodiment, one or more of the tubes 100, 110, 120 can have a sensor or other tracking device 1900. The tracking device 1900 can be used to confirm the position of the active cannula 10 within the targeted anatomical region. It should be understood by one of ordinary skill in the art that any or all of the tubes can have a tracking device 1900. For example, proper positioning and orientation of the first tube 100 can be verified by the tracking device 1900 so that the remaining tubes can be extended therefrom in sequence. In one embodiment, the tracking device 1900 can be an electromagnetic tracking device that is used with a monitor 2000 (shown in FIG. 20). The electromagnetic tracking can determine the position and orientation of the one or more tubes using electromagnetic coils on one or more of the tubes to detect EM field strength. Exemplary components that can be utilized are available from TRAXTAL™ or AURORA™. As another example, optical tracking components can be utilized, such as the NDI Optotrak Certus Motion Capture System. Other techniques and components can be used as a location sensor or transmitter and a location monitor or receiver, including ultrasound components.

Where the tubes of the active cannula are made from SMP, a number of manufacturing techniques can be utilized. As described above, the SMP tubes can be pre-shaped and preserved in the same shape prior to intervention. In another embodiment, the SMP tubes can be pre-shaped at higher temperatures (e.g., +75° C.), cooled down to room temperature (+20-25° C.) where they take prior shape (e.g., straight), and re-shaped at the beginning of surgery, such as by introducing tubes into a warm fluid (e.g., sterilization fluid). In another embodiment, the SMP tubes can be pre-shaped as described above but at lower temperatures (e.g., +37° C.), cooled down to room temperature, and then reshaped inside of the body, such as by using body temperature as a transition inducer. SMA materials with higher transition materials would be difficult to form using some of these above described techniques.

Active cannula 10 allows a user (e.g., a physician) to overcome difficulties created by the small size of the cannulas that are desired in minimally invasive procedures. Achieving correct orientation can be difficult, inaccurate and time consuming. The length of each tube is typically intended to be precise, both in absolute terms and relative to the other tubes to enable correct extension. Manual assembly can be difficult and time consuming. Tubes are hard to handle, particularly at the smaller sizes (e.g. 0.007 inches or 0.778 mm). Deployment requires that the tubes maintain their relative orientation while being advanced into the patient. Precise deployment can also be difficult. Maintaining the correct orientation of each tube with respect to the other tubes as they are being deployed can be error-prone. It can be difficult to grasp the very small tubes and manual advancement can be imprecise without mechanical assistance. Active cannula 10 can set the correct length and orientation of each nested sub-tube into a lockable block or support structure. Each block can be mounted into or otherwise provided to a frame that maintains orientation as well as distance. The precise setting of the blocks on the tubes within the frame assures that the sequential deployment of the tubes will reach the correct target, while traversing a very specific path. The active cannula 10 can be used in various procedures and various portions of the body, including the lungs, brain, heart, gall bladder and so forth. Other uses are also contemplated by the present disclosure.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific embodiments have been illustrated and described herein, it should be appreciated that any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description. Therefore, it is intended that the disclosure not be limited to the particular embodiment(s) disclosed as the best mode contemplated for carrying out this invention, but that the invention will include all embodiments falling within the scope of the appended claims.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims.

What is claimed is:

1. A device for accessing a targeted anatomical region of a body, the device comprising:
a plurality of hollow tubes (100, 110, 120);
a plurality of blocks (200, 210, 220), wherein each of the blocks is connected to one of the hollow tubes; and
a track (1800), wherein each of the blocks is operably connected to the track for movement therealong, wherein in a first position the blocks are separated from each other along the track and the plurality of hollow tubes are nested, wherein in a second position the blocks are adjacent to each other along the track and the plurality of hollow tubes are extended, and wherein in the second position the plurality of hollow tubes provides access to the targeted anatomical region from outside of the body.

2. The device of claim 1, wherein each of the blocks (200, 210, 220) is rigidly connected to one of the hollow tubes (100, 110, 120) to prevent axial or rotational movement of the hollow tube with respect to the block.

3. The device of claim 1, wherein the track (1800) restrains movement of the plurality of blocks (200, 210, 220) in all but two directions, and wherein the two directions are opposite to each other.

4. The device of claim 1, wherein at least a portion of the plurality of hollow tubes (100, 110, 120) are made from a shape memory alloy.

5. The device of claim 4, wherein the shape memory alloy is nickel titanium.

6. The device of claim 1, wherein the innermost tube of the plurality of hollow tubes (100, 110, 120) has an inner diameter large enough for passing a surgical device therethrough.

7. The device of claim 1, further comprising a location sensor (1900) that provides a location signal.

8. A system for accessing a targeted anatomical region of a body, the system comprising:
a plurality of support structures (200, 210, 220);
a plurality of tubes (100, 110, 120) that are each connected to one of the support structures; and
a guide (1800), wherein each of the support structures are operably connectable with the guide, wherein the guide allows movement of at least a portion of the plurality of support structures therealong, wherein the plurality of tubes are nested when the plurality of support structures are in a first position along the guide, wherein the plurality of tubes are extended when the plurality of support structures are in a second position along the guide, and wherein in the second position at least one of the plurality of tubes accesses the targeted anatomical region of the body.

9. The system of claim 8, further comprising a configuration device (300) that connects the plurality of tubes (100, 110, 120) to the support structures (200, 210, 220) at a desired length and orientation of the tube with respect to the support structure.

10. The system of claim 9, wherein the configuration device (300) has a calibration mechanism (360).

11. The system of claim 8, wherein the guide (1800) allows movement of at least a portion of the plurality of support structures (200, 210, 220) in only two directions that are opposite to each other.

12. The system of claim 8, wherein at least a portion of the plurality of tubes (100, 110, 120) are made from a shape memory alloy.

13. The system of claim 12, wherein the shape memory alloy is nickel titanium.

14. The system of claim 8, wherein each of the plurality of tubes (100, 110, 120) are hollow, and wherein the innermost tube of the plurality of tubes has an inner diameter large enough for passing a surgical device therethrough.

15. The system of claim 8, wherein in the second position each of the plurality of support structures (200, 210, 220) abut against each other along the guide (1800).

16. The system of claim 8, further comprising a location transmitter (1900) connected to at least one of the plurality of tubes (100, 110, 120) and a receiver (2000) for receiving a location signal from the location transmitter.

17. A method for accessing a targeted anatomical region of a body, the method comprising:
determining a path to the targeted anatomical region;
providing a plurality of tubes (100, 110, 120) having a length and shape to follow the path;
connecting each of the tubes to support structures (200, 210, 220);
positioning the support structures so the tubes are in a nested position; and
moving the support structures so the tubes are in an extended position and a portion of the plurality of tubes is in proximity to the targeted anatomical region.

18. The method of claim 17, further comprising connecting each of the tubes (100, 110, 120) to the support structures (200, 210, 220) by rigidly fixing a length and orientation of the tubes with respect to the support structures.

19. The method of claim 17, further comprising moving the support structures (200, 210, 220) to abut against each other so the tubes (100, 110, 120) are in the extended position.

20. The method of claim 17, further comprising moving the support structures (200, 210, 220) so the tubes (100, 110, 120) are in another extended position and a portion of the tubes is in proximity to another targeted anatomical region.

21. The device of claim 1, wherein at least a portion of the plurality of hollow tubes (100, 110, 120) are made from a shape memory polymer.
